(19) United States
(12) Patent Application Publication
(10) Pub. No.: US 2010/0239669 A1
(43) Pub. Date: Sep. 23, 2010

(54) Combinations of statins and anti-obesity agent

(75) Inventor: Negeswara R. Palepu, Southampton, PA (US)

Correspondence Address: Irving M. Fishman, 89 Headquarters Plaza, Suite 1422, North Tower, Morristown, NJ, 07960, US

(73) Assignee: Scidose LLC, Amherst, MA (US)

(21) Appl. No.: 12/800,732

(22) Filed: May 21, 2010

Related U.S. Application Data
(62) Division of application No. 12/080,950, filed on Apr. 7, 2008.
(60) Provisional application No. 60/922,454, filed on Apr. 9, 2007.

(51) Int. Cl. A61K 9/28; A61K 31/40; A61K 31/366; A61K 31/405; A61K 31/22; A61K 31/505; A61K 31/365; A61P 3/04; A61P 9/12; A61P 3/06

(52) U.S. Cl. 424/474; 514/423; 514/460; 514/415; 514/548; 514/275; 514/449

(57) ABSTRACT

Co-therapy of an anti-obesity agent, a statin, is disclosed along with fixed combinations thereof. Atorvastatin and orlistat are preferred as the various components.

### CROSS-REFERENCE TO RELATED APPLICATIONS

This application claims benefit of U.S. Provisional Application Ser. No. 60/922,454, filed Apr. 9, 2007.

### STATEMENT REGARDING FEDERALLY SPONSORED RESEARCH OR DEVELOPMENT

Not Applicable

### FIELD OF THE INVENTION

The present invention relates to the field of statin therapeutic agents; to the field of anti-obesity agents (such as orlistat and sibutramine); to combination therapy utilizing them together, either as separate administration of separate formulations or together; and most preferably as single fixed combination products. The invention further relates to improved methods of reducing serum triglyceride and/or cholesterol and/or weight reduction, and especially enhanced reduction in one or both of serum triglyceride and/or serum cholesterol than can be achieved with the individual agents. The invention further relates to combination therapy which allows for reduction of the dosages of the individual agents below those levels at which they would be used in monotherapy to achieve the same or substantially the same results.

### BACKGROUND OF THE INVENTION

Various statins have been found to be effective HMG-CoA reductase inhibitors. Statins that are currently available for treating hyperlipidemia and/or hypercholesterolemia include atorvastatin (Lipitor® from Pfizer), simvastatin (Zocor® from Merck), pravastatin (Pravachol® from Bristol Myers Squibb), fluvastatin (Lescol® from Novartis), lovastatin (Mevacor® from Merck), and rosuvastatin (Crestor® from AstraZeneca). The anti-obesity component, orlistat (Xenical® from Roche) works by inhibiting the absorption of fats from the gastrointestinal tract (and thereby prevents the body from utilizing the unabsorbed fats in multiple processes, including the biosynthesis of cholesterol and for losing weight) or sibutramine (Meridia® from Abbott) works centrally via reuptake inhibition of norepinepherine, dopamine, and serotonin. The statins have a very different mechanism of action in that they are HMG CoA reductase inhibitors and therefore interfere in the conversion of one intermediate in the cholesterol biosynthetic pathway into another.

### OBJECTS OF THE INVENTION

It is therefore an object of the invention to provide a method of enhancing the effectiveness of statins by administering to a patient in need thereof co-therapy which includes at least one statin in combination with at least one anti-obesity agent.

It is another object of the invention to provide a composition comprising at least one statin and at least one anti-obesity agent.

It is still another object of the invention to provide a synergistic composition comprising (a) at least one statin and (b) at least one anti-obesity agent.

Yet another object of the invention is to provide a method of achieving a reduction in cholesterol and/or triglycerides in a patient in need thereof that is in excess of such reductions achievable with monotherapy with either a statin or at least one anti-obesity agent.

Still another object of the invention is to provide a statin co-therapy with an anti-obesity agent where the statin is atorvastatin or a pharmaceutically acceptable salt thereof.

Still another object of the invention is to provide a statin co-therapy with an anti-obesity agent where the anti-obesity agent is orlistat or a pharmaceutically acceptable salt thereof.

Still another object of the invention is to provide a statin co-therapy with an anti-obesity agent where the anti-obesity agent is sibutramine or a pharmaceutically acceptable salt thereof.

Even further object of the invention will be apparent to those of ordinary skill in the art.

### BRIEF SUMMARY OF THE INVENTION

These and other objects of the invention can be achieved in patients in need of cholesterol and/or serum triglyceride reduction or control and/or weight reduction or control by treating such patients with a co-therapy comprising at least one statin and at least one anti-obesity agent. Preferably, the co-therapy is via a dosage form having both of the agents in a single dosage form.

### BRIEF DESCRIPTION OF THE SEVERAL VIEWS OF THE DRAWING

Not Applicable

### DETAILED DESCRIPTION OF THE INVENTION

The present invention is a combination of at least one statin, and at least one anti-obesity agent, whether in a single dosage form or administered in separate dosage forms each having one of the two active agents (statin and anti-obesity agent), either simultaneously, sequentially, or at different times of the day. In addition to the these agents mentioned above, additional active agents can be optionally added to the co-therapy regimen, whether as additional standalone products or as fixed combination products with any or all of the other statin and/or anti-obesity agents. Whenever an active agent is referred to herein, it includes the free compound named and its various pharmaceutically acceptable salts. Mention of the compound name, without reference to polymorphic form or crystallinity or lack thereof includes amorphous and crystalline forms of any kind. Reference is made to U.S. application Ser. No. 11/282,507, filed Nov. 18, 2005, incorporated by reference in its entirety for one manner of making non-crystalline forms. Mention of the compound name without reference to solvate or non-solvate includes hydrates, anhydrous forms, other solvates, unsolvated forms, and mixed solvates (a hydrate being a solvate where the solvent molecule is water).

In the simplest form of the invention the anti-obesity component, the statin component, and any optional additional agent are each in different dosage forms. In this aspect, the currently marketed forms of these agents is suitable and they may be used in amounts that range from the below the minimally effective amounts as set forth in their respective labeling as of the filing date of this application (i.e., taking benefit of the synergistic results of the invention) to a maximum of their respective maximum tolerated dosage, generally not in excess of twice the maximum recommended amounts as indicated in their respective labeling as of the filing date of the present application. The co-therapy of the invention yields results that would not be achievable with the entity as monotherapy even beyond the maximum tolerated dose of the active agents. Preferably, the maximum tolerated dosage of the individual agents is not used and the preferred maximum amount of each agent is within the maximum recommended dosages in their respective labeling as of the filing date of the present application. There is no set ratio of one component to the other within the above amounts that is not or should not be considered for use, all of them being within the current invention. For the respective compounds which are not currently marketed, the range of dosages for consideration in the present invention should be that amount which gives approximately equal therapeutic responses on average to its closest marketed related compound in at least one indication for its closest marketed related compound as of the filing date of the present application. Thus, if an unmarketed “atorvastatin-like drug” is used as the statin, its range of dosages for the present invention should be based on either atorvastatin (currently marketed in the US) or to a more closely related statin that is currently marketed elsewhere in the world. Of course, if the compound is marketed elsewhere (i.e. other than the US) as of the filing date of the present application but not in the US, then the dosage should be calculated based on that marketed labeling. Where the US dosage range and the dosage range in labeling from other countries differ, the lowest minimum and the highest maximum (not necessarily being in the same label) should be considered as the “currently marketed dosage range”. Similar guidelines should be used for the dose calculation of, the anti-obesity agents. Additional active agents that are desirable to coadminister and are included in the co-therapy or co-formulation should generally be used in the dosage ranges recommended in their respective labeling when those additional actives are otherwise used as standalone therapy.

The statins belong to a group of compounds that have the following formula I

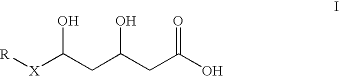

where X is straight or branched —(CH₂)ₘ— or —CH═CH—, preferably —(CH₂)ₘ—; with m being 0-4 (preferably 1) or its corresponding lactone of formula II

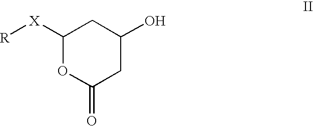

where R in each case is a 5-6 membered monocyclic or 9-10 membered bicyclic group which may be substituted with a variety of substituents. For purposes of the present invention, the term “statin also includes (unless specifically restricted otherwise or the context requires restriction) the pharmaceutically acceptable salts and esters of the acid group shown in Formula I above. Typical statins that are commercially available include: atorvastatin, fluvastatin, lovastatin, pravastatin, rosuvastatin, and simvastatin.

R in formula I may be selected from the group of formulas III, IV, V, and VI; where formula III is

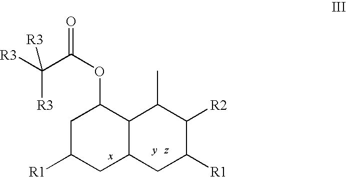

where R1-bond x-bond y-bond z-R1 represents R1-C*H—CH═C*—CH═C*—R1, R1-C*H—CH═C*—CH₂C*H—R1, R1-C*H—CH₂—C*═CH—C*H—R1, or R1-C*H—CH₂—C*H—CH═C*—R1; where * indicates a bond to the rest of the structure (in other words either (1) one of bonds x, y, and z is a double bond or (2) y is a single bond and both of x and z are double bonds);
each R1 being independently selected form H, OH, or alkyl of 1-4 carbon atoms (preferably of 1 carbon);
R2 being selected form H or alkyl of 1-4, preferably 1, carbon atom;
each R3 being independently selected from H and alkyl of 1-4 carbons, preferably of 1 carbon;

where formula IV is

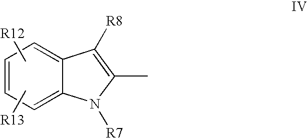

in which one of R7 and R8 is a phenyl ring optionally having from 1-3 substituents independently selected from selected from alkyl of 1-4 carbons, alkoxy of 1-4 carbons, halogen (preferably fluoro or chloro), phenoxy, and benzyloxy; the other of R7 and R8 is a primary or secondary alkyl of 1-5 carbons; and each of R12 and R13 is independently selected from H, straight or branched chain alkyl of 1-4 carbons, straight or branched alkoxy of 1-4 carbons, cycloalkyl of 3-6 carbons, trifluoromethyl, fluoro, chloro, phenoxy and benzyloxy;

where formula V is

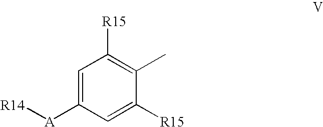

where A is S, —SO₂—, or N, the N being optionally substituted by straight or branched alkyl of 1-5 carbon atoms (preferably methyl);
R14 is selected from (1) alkyl of 1-3 carbons (preferably methyl), optionally substituted by 1-3 substituents selected from halogen, amino, and/or cyano, (2) an aromatic group of 6-12 carbons optionally substituted by 1-3 substituents selected form alkyl of 1-3 carbons, halogen, amino, or cyano, or (3) alkyl of 1-3 carbons (preferably methyl), optionally substituted by 1-3 substituents independently selected from an aromatic group of 6-12 carbons which is further optionally substituted by 1-3 substituents selected form alkyl of 1-3 carbons, halogen, amino, or cyano;
each of R15 is independently selected from (1) H, (2) alkyl of 1-3 carbons optionally substituted by halogen, amino, and/or cyano, and (3) an aromatic of 6-12 carbons (preferably phenyl) optionally substituted by alkyl, halogen (preferably fluoro), and/or amino;

where formula VI is

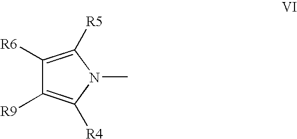

where R4 is selected from straight or branched alkyl of 1-6 carbons, cycloalkyl of 3-6 carbons, and trifluoromethyl;
R5 is selected from 1-naphthyl, 2-naphthyl, cyclohexyl, norbornyl, or phenyl (optionally substituted with fluorine, chlorine, bromine, hydroxyl, trifluoromethyl, alkyl of 1-4 carbons, alkoxy of 1-4 carbons, or alkanoyloxy of from 2-8 carbons);
either of R6 or R9 is —CON(R10)(R11) in which R10 and R11 are each independently selected from hydrogen, alkyl of 1-6 carbons, or phenyl optionally substituted with fluorine, chlorine, bromines, cyano, trifluoromethyl and/or carboalkoxy of 3-8 carbon atoms;
and the other of R6 and R9 is selected from hydrogen, alkyl of 1-6 carbon atoms, cyclopropyl, cyclobutyl, cyclopentyl, cyclohexyl, or phenyl, which phenyl is optionally substituted with fluorine, chlorine, broine, hydroxyl, trifluoromethyl, alkyl of 1-4 carbons, alkoxy of 1-4 carbons, and/or alkanoyloxy of 2-8 carbons;

Atorvastatin and atorvastain-like drugs are of formula VI above and are described more specifically, including the manner of making and using them, in one or more of U.S. Pat. Nos. 4,681,893; 5,273,995; 5,686,104; 5,969,156; and 6,126,971, all of which are incorporated herein by reference in their entireties. In some embodiments, the atorvastatin or atorvastatin-like drug is in the form of its calcium salt. “Atorvastatin” as the free compound is specifically the compound (βR,δR)-2-(4-fluorophenyl)-beta,delta-dihydroxy-5-(1-methylethyl)-3-phenyl-4-[(phenylamino)carbonyl]-1H-pyrrole-1-heptanoic acid.

Simvastatin and simvastatin-like drugs belong to formula III above and are described more specifically, including the manner of making and using them, in one or more of U.S. Pat. Nos. 4,444,784; RE36481; and RE36520, all of which are incorporated herein by reference in their entireties.

Pravastatin and pravastatin-like drugs belong to formula III above and are described more specifically, including the manner of making it and using it, in one or more of U.S. Pat. Nos. 4,346,227; 5,030,447; 5,180,589; and 5,622,985; all of which are incorporated herein by reference in their entireties.

Fluvastatin and fluvastatin-like drugs belong to formula IV above and are described more specifically, including the manner of making and using them, in one or more of U.S. Pat. Nos. 5,354,772; and 5,356,896, each of which is incorporated herein by reference in their entireties.

Lovastatin and lovastatin-like drugs belong to; formula III above and are described more specifically, including the manner of making and using them, in U.S. Pat. No. 4,231,938, which is incorporated herein by reference in its entirety.

Rosuvastatin and rosuvastatin-like drugs belong to formula V above and are described more specifically, including the manner of making and using them, in one or more of U.S. Pat. Nos. 6,316,460; 6,589,959; and RE 37,314, all of which are incorporated herein by reference in their entireties.

The anti-obesity agent for use in the present invention is selected from orlistat and sibutramine-type compounds.

Orlistat is the N-formyl-L-leucine ester of (3S,4S)-3-hexyl-4-[(2S)-2-hydroxytridecyl]-2-oxetanone, and has the structure

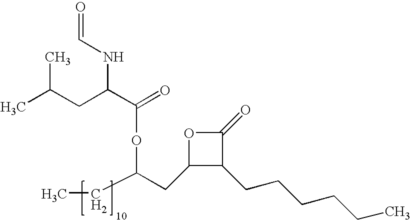

It is commercially available from Roche under the name XENICAL, and is described in detail, including the manner of making and using it in U.S. Pat. No. 4,598,089, which is incorporated herein by reference in its entirety.

Sibutramine type compounds are of the following formula VIII

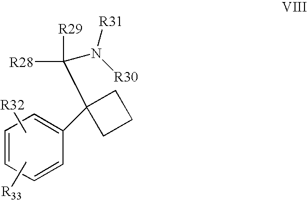

where R28 is a branched alkyl of up to 6 carbons, R29 is H or an alkyl of 1 to 3 carbons, R30 and R31 are the same or different and selected from H, straight or branched alkyl of 1 to 4 carbons, alkenyl of 3 to 6 carbons, alkynyl of 3 to 6 carbons, cycloakyl of 3 to 7 ring members, or formyl, and R32 and R33 are the same or different and selected from H, halo, trifluoromethyl, alkyl of 1 to 3 carbons, alkylthio of 1 to 3 carbons, and phenyl or R32 and R33 together with the carbon atoms to which they are attached form a second benzene ring, which second benzene ring is optionally substituted by (a) at least one halo, alkyl, or alkoxy group containing 1 to 4 carbons, or (b) the substituents of the second benzene ring together with the carbon atoms to which they are attached form a third benzene ring as well as pharmaceutically acceptable salts thereof. Sibutramine itself has the structure

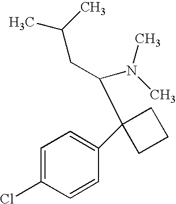

Sibutramine type compounds are discussed in further detail, including the manner of making and using them in U.S. Pat. No. 4,746,680, U.S. Pat. No. 4,929,629, and U.S. Pat. No. 5,436,272, all of which are incorporated herein by reference in their entirety. Of the sibutramine type compounds, sibutramine and its pharmaceutically acceptable salts are preferred. Sibutramine is available in 5 mg, 10 mg, and 15 mg oral capsules and is recommended for use at doses of 5 mg to 15 mg once daily. For the purposes of the present invention, sibutramine can be used at dosages of about 1 mg once daily to about 30 mg once daily.

Of the above statins, atorvastatin, fluvastatin, lovastatin, pravastatin, simvastatin, and rosuvastatin, or pharmaceutically acceptable salts thereof (or the lactone or the non-lactone variants thereof as applicable) are preferred, in part because they are in commercial medical use. Of these, atorvastatin, its pharmaceutically acceptable salts, and the lactone version thereof is more highly preferred. Of the atorvastatin salts, amino acid, sodium and calcium salts are preferred, with calcium salts being more highly preferred.

Orlistat is used in current approved labeling in amounts of 120 mg three times a day with meals containing fat for weight reduction purposes. For the present invention, the orlistat is used in amounts of from 30 mg once daily up to 480 mg per day in divided doses, generally up to 120 mg three times daily. The purpose of the orlistat in the combination therapy of the present invention is not weight reduction per se, but weight reduction can be an added benefit. Rather, the intended purpose of the orlistat is to reduce the total absorbed fat levels that are otherwise absorbed so as to limit bioavailable fat from the diet for purposes of cholesterol biosynthesis and thus to complement and boost the effectiveness of the statin in question.

Atorvastatin is currently recommended in its current US labeling for cholesterol reduction at doses of 10 mg to 80 mg once daily. For purposes of the present invention, it can be used at dosages as low as 2.5 mg up to 160 mg once daily or in divided doses, generally up to 80 mg once daily or in divided doses.

Lovastatin is currently recommended to be administered in its current US labeling in amounts of 10 mg to 80 mg once daily or in 2 divided doses. For purposes of the present invention lovastatin can be used at doses as low as 2.5 mg up to 160 mg once daily or in divided doses, generally up to 80 mg once daily or in divided doses.

Fluvastatin is recommended to be administered in its current US labeling in doses of from 20 mg to 80 mg once daily or in divided doses. The present invention allows for fluvastatin to be doses at 5 mg daily up to 160 mg once daily or in divided doses, generally up to 80 mg daily in single or divided doses.

Pravastatin is recommended for administration in its current US marketed label in amounts of 10 mg to 80 mg once daily. The present invention allows for the use of pravastatin at a dose as low as 2.5 mg daily up to 160 mg once daily or in divided doses, generally up to 80 mg daily.

Simvastatin is recommended in its current US label at doses of 5-80 mg once daily. The present invention permits dosing of simvastatin at 1.25 mg daily 160 mg once daily or in divided doses, generally up to 80 mg daily.

Rosuvastatin, when used for hypercholesterolemia control is dosed at 5 mg to 40 mg, once daily. The present invention permits dosing of rosuvastatin at about 1 mg daily to about 80 mg once daily or in divided doses, generally up to 40 once daily or in divided doses.

The ratio of the anti-obesity agent to the statin can be any ratio that permits the anti-obesity agent and the statin to be administered within the ranges set forth above; however, most preferable for ease of use of the currently marketed standalone products are ratios which use a currently marketed dosage form of each active. Thus, for example, on a daily basis, 120 mg of orlistat is preferred in one embodiment and this is paired up with a currently marketed dosage form of one of the marketed statins and if desired, a currently marketed dosage form of another active agent. These same dosages in particular fixed combination dosage forms of 2 or more of the above agents are advantageous in that it permits ready titration of patients and then conversion to the fixed combination. Other fixed combinations are advantageous as they can fill the gaps in the dosing steps needed to change patients between dosages or to individualize treatment regimes to the patient.

Sample fixed combination dosages are set forth below for atorvastatin and orlistat. Similar ratios for the other marketed statins and the other marketed anti-obesity agents will be apparent to those of ordinary skill. Dosages for other statins and anti-obesity agents that are not the specific ones set forth above but are within the formulas above can be calculated as:
unmarketed statin compound minimum for the invention=¼ of an amount that is approximately equal therapeutic response to the minimum of the closest marketed statin
unmarketed statin compound maximum for the invention=maximum tolerated dose of the unmarketed statin compound
unmarketed anti-obesity compound minimum for the invention=¼ of an amount that is approximately equal therapeutic response to the minimum of the closest marketed anti-obesity agent
unmarketed anti-obesity agent compound maximum for the invention=maximum tolerated dose of the unmarketed anti-obesity agent compound.
   Use of sibutramine in place of the orlistat merely replaces the 120 mg OD, 120 mg BID, and 120 mg TID in the table below with sibutramine 5 mg OD, 10 mg OD or 5 mg BID, and 15 mg OD or 5 mg TID, respectively.

Sample (non-limiting) combination dosages (free combination of marketed dosage forms)*:

| | | |
|---|---|---|
| Atorvastatin | 120 mg OD | 10 mg OD |
| Lovastatin | | |
| Pravastatin | | |
| Or Simvastatin | | |
| | 120 mg BID | 10 mg OD |
| | 120 mg TID | 10 mg OD |
| | 120 mg BID | 10 mg BID |
| | 120 mg TID | 10 mg TID |
| | 120 mg OD | 20 mg OD |
| | 120 mg BID | 20 mg BID |
| | 120 mg TID | 20 mg TID |
| | 120 mg OD | 40 mg OD |
| | 120 mg BID | 40 mg BID |
| | 120 mg TID | 40 mg BID |
| | 120 mg OD | 80 mg OD |
| | 120 mg BID | 80 mg OD |
| | 120 mg OD | 20 mg OD |
| | 120 mg TID | 40 mg BID |
| | 120 mg BID | 80 mg OD |
| | 120 mg TID | 80 mg OD |
| Simvastatin | 120 mg OD | 5 mg OD |
| | 120 mg BID | 5 mg BID |
| | 120 mg TID | 5 mg TID |
| Fluvastatin | 120 mg OD | 20 mg OD |
| | 120 mg BID | 20 mg BID |
| | 120 mg BID | 20 mg BID |
| | 120 mg BID | 40 mg BID |
| | 120 mg BID | 40 mg BID |
| | 120 mg TID | 20 mg TID |

* OD = once daily; BID = twice daily; TID = three times daily. Free combinations where each component is administered on the same schedule can also be administered as fixed combination products of all three components.

Sample combination dosages at dosages below the minimum commercially available dosages of the various products include, without limitation:

| | Orlistat Dose | Statin Dose | |
|---|---|---|---|
| Atorvastatin | 30 mg | 5 | mg |
| Lovastatin | | | |
| Pravastatin | | | |
| Or Simvastatin | | | |
| | 60 mg | 5 | mg |
| | 90 mg | 5 | mg |
| | 30 mg | 10 | mg |
| | 60 mg | 10 | mg |
| | 120 mg | 10 | mg |
| | 30 mg | 5 | mg |
| | 60 mg | 5 | mg |
| | 90 mg | 5 | mg |
| | 30 mg | 10 | mg |
| | 60 mg | 10 | mg |
| | 90 mg | 10 | mg |
| | 30 mg | 5 | mg |

Fixed combination dosage forms can be prepared in any manner known in the art and are especially prepared from the materials that are utilized in the formulation of the standalone single active agent corresponding products. They may be made by blending the active agents together in a single blend, or preparing pre-blends of less than all of the active agents and forming each into separate granulations for blending together, or the actives can individually be prepared into beads for blending and filling into capsules or compression into tablets. In other formats, one or more of the active agents can be formulated as a separate portion of the dosage form as in the case of bi-layered or tri-layered tablets. Those of ordinary skill in the art will be aware of further variations on the theme.

In addition to the above, it should be noted that one or more of the active agents can be administered by alternative routes of administration, i.e., non-oral routes for any of the actives other than the orlistat. Thus, oral orlistat combined with a transdermal administration of the statin for example is also within the present invention. Those of ordinary skill will be aware of further alternate routes by which the statin and other anti-obesity agents can be administered. Particularly advantageous formulations for atorvastatin or atorvastatin containing fixed combinations are set forth more fully below.

In each of the above embodiments, whether separate agents in separate dosage forms, or fixed combinations, one or more further active agents can also be added to the co-therapy regimen. These further agents can be added in free combination with the above or may also be in fixed combination with one or more of the other agents. For example, in a three active agent scenario, (a) each of the active agents 1, 2, and 3 may be used in free combination, or (b) agents 1 and 2 may be in fixed combination with each other and used in free combination with agent 3, or agents 1 and 3 may be used in fixed combination with each other and used in free combination with agent 2 or agents 2 and 3 may be in fixed combination with each other and used in free combination with agent 1 or (c) all of agents 1, 2, and 3 are in fixed combination with each other. Those of ordinary skill in the art will appreciate the various alternatives when still further active agents are added to the co-therapy. Any route of administration for the active agents is suitable provided such route is compatible with both the active agent per se and the activity for which that agent is intended to deliver. Thus, when orlistat is used, the orlistat containing product should be administered orally as orlistat action is in the GI tract. The statins, sibutramine, and the other optional agents used in the present invention co-therapy are, however, not so limited.

Inactive agents which can be used are any of those that are compatible with the active agents that are in contact therewith and are pharmaceutically acceptable. These are generally known in the art (both components and relative amounts and specifically indicated in the various patents set forth herein, all of which are incorporated herein in their entirety by reference. These typically include, without limitation, active agent stabilizers (inclusive of chemical stabilizers and physical stabilizers, etc.), diluents, binders, disintegrants, surfactants, lubricants, glidants, and coating materials. Any of the inactive agents present in the currently marketed products containing the respective active agent may be used for that component of the fixed combination products of the present invention and unless there is an incompatibility that results with the other active agents in the invention fixed combinations, may be used in intimate contact with the other active agent as well.

Where single granulations contain more than one active agents, then the inactives need to be compatible with each of the active agents, Since coating materials are not in intimate contact with the active agents, they may, in some instances have some incompatibilities with the active agents, and if so, then it is preferably to have an intermediary barrier coating that separates the incompatible coating components form the remainder, but if acceptable formulation stability in the absence of such intermediary barrier is obtained, the barrier layer need not be used. Those of ordinary skill will be able to select the appropriate coating materials based on simple testing or knowledge already available in the art.

Typical preferred inactive agents include, without limitation, bulking agents (for example without limitation, mono and disaccharides (such as dextrose, lactose, sucrose, etc.), sugar alcohols (such as mannitol, xylitol, sorbitol. etc.) and other bulking agents (such as microcrystalline cellulose, dicalcium phosphate, tricalcium phosphate, etc.)), surfactants (such as polyethyleneglycols, polyethylene glycol/polypropylene glycol block or random compolymers, Tweens, Vitamin E TPGS, Tween surfactants, Brij surfactants, fatty alkyl sulfates, fatty alkyl sulfonates, polyethoxylated fatty alkyl sulfates, polyethoxylated fatty alkyl sulfonates, etc.), binders (such as hydroxypropylcellulose, hydroxypropylmethylcellulose, hydroxyethylcellulose, povidone, carboxymethylcellulose, sodium carboxymethylcellyulose, etc.), disintegrants and superdisintegrants (such as povidone, crospovidone, croscarmellose sodium, sodium starch glycollate, etc,), alkalinizing salts such as (alkali metal or alkaline earth metal salts of carbonate or bicarbonate or silicate, alkaline earth metal hdyoxide, magnesiumaluminum silicate, magnesium aluminum hydroxide, etc.), lubricants and glidants (such as alkali metal or alkaline earth metal salts of fatty acids, silicon dioxide, talc, etc.), and typical coating agents known in the art. The typical coating agents can be mere film coatings that do not alter dissolution profiles (for example, without limitation, those available under the OPADRY name), those that delay release that are either pH dependent or pH independent, and those that impart controlled or sustained release. Each of the inactive agents can vary over wide ranges in terms of the percent of the formulation that they make up and is in part dependent upon the amount of active agent being administered and the particular dissolution profile being sought. A highly preferred formulation is set forth in the Examples, but a wide range of other compositions are suitable as well.

Dosage form construction can be along the lines of single granulation, with one or more of the active agents in the granule or one or more of the active agents intragranularly and one or more of the active agents extragranularly, or one or more of the active agents can be coated or adsorbed onto or into a carrier particle. Alternatively, one or more of the active agents may be included into an oral-osmotic dosage form of the type that has become known as OROS formulations many of which have been patented by ALZA Corporation in the 1970s and 1980s. Alternatively, bilayer or multilayer formulations may be prepared where the active agents may be in the same or different layers and the different layers may have similar or different physical functions with respect to release rates such as rapid swelling to allow for gastric retention of all or part of the dosage form in the stomach for release of one or more of the active agents in the stomach (such as for example, without limitation, those patented by Jagotech or by Depomed). A further alternative is to have a capsule dosage form (whether hard or soft) containing the various active agents either as granulates or in the form of minitablets, with or without extragrnaular inactive agents or extragranular active agent as well. Still other dosage form constructions for fixed combinations will be apparent to those of ordinary skill in the art.

In a particularly preferred embodiment, a statin active agent is blended with a superdisintegrant such as croscarmellose sodium and optionally microcrystalline cellulose. This blend is granulated with an aqueous solution or dispersion of a surfactant like material such as Vitamin E TPGS, which granules are then sieved and dried. The dried granules are then blended with the anti-obesity agent, a carrier such as lactose, microcrystalline cellulose, a disintegrant such as croscarmellose sodium or sodium starch glycollate, and either or both of a lubricant and glidant. The blend is then compressed into a single tablet. Alternatively, the anti-obesity agent may be incorporated into the granule by blending part or all of it with the other intragranular components before granulation. Similarly, a portion or all of the statin active agent can be in the extragranular portion.

Additional active agents can be added as an intragranulate component of the statin granulate, an intragranulate component of the anti-obesity component granulate or if desired it can be added extragranularly. Design choices such as the individual active agent pharmacokinetics will help guide the choice, but any arrangement is within the scope of the present invention. Generally, most active agents will be at least partially within the statin granulate or anti-obesity granulate, or intragranulate component of the statin and anti-obesity active agent containing granulate. Alternatively, the additional active agents may be formulated in their own granulates which are blended with the granulate or granulates containing one or both of the statin and the anti-obesity active agent.

Additional processes may include colyophilization of the two medicaments or any with or without surfactant or solubilizer and with or without an internal disintegrant. The lyophilized blend is then mixed with bulking excipients and disintegrant, lubricated and compressed into tablets or filled into capsules. A binder can also be used in the colyophilization.

Exemplary formulations are set forth in the examples appended hereto. Using the formulations in Example 3 there and the statin as the active agent alone as a base formulation (i.e. an 80 mg atorvastatin standdalone formulation, that is without the other orlistat of the examples), the formulation can have the other active agents added intragranularly by replacing a portion of the intragranular and/or extragranular microcrystalline cellulose and/or extragranular lactose or simply be added to the base composition intragranularly. The additional optional active agents can be added alternatively as their own granulate or extragranularly as desired, generally by replacing a portion of the extragranular microcrystalline cellulose and/or lactose. When used extragranularly, they can be added in partial replacement of the extragranular microcrystalline cellulose and/or lactose, or simply added without replacement of any of the microcrystalline cellulose or lactose. In this manner, each of the 80 mg atorvastatin containing compositions can be obtained with the additional required and/or optional active agents of the co-therapy in fixed combinations thereof. For lower dose atorvastatin, one can either start with a proportional amount of the 80 mg atorvastatin base formulation mentioned above (i.e., ⅛th for a 10 mg formulation) or start with the base formulation set forth above except using a lesser amount of the atorvastatin (i.e., simply replace the 80 mg atorvastatin with 10 mg atorvastatin in the otherwise base formulation referred to above) and include the other active agents as indicated above concerning the 80 mg containing combinations. In each of these, the atorvastatin may be replaced by appropriate amounts of the other statins to arrive at formulations containing those statins. Furthermore, in each case, the microcrystalline cellulose and lactose can be replaced in whole or in part by other pharmaceutically acceptable bulking agents such as, without limitation, those as set forth previously, and the croscarmellose sodium and sodium starch glycolate can be in whole or part replaced by other pharmaceutically acceptable disintegrants, such as, without limitation, those as set forth above, and the magnesium stearate can be replaced in whole or part by other pharmaceutically acceptable lubricants and/or glidants, such as, without limitation, those as set forth above. In each of the formulations thus arrived at (which are the most preferred amounts), the ranges of the inactive components can vary from those derived from the above (to arrive at still preferred, but not most preferred amounts) as follows: the bulking agents can be +/− about 15% of the amounts otherwise arrived at; the disintegrants can be +/− about 15% of the amounts otherwise arrived at; the lubricants/glidants can be +/− about 2% of the amounts otherwise arrived at, and the TPGS component should be at a minimum of about 5 mg in any formulation and can vary up to about 40 mg in any formulation otherwise arrived at. Notwithstanding the above, even broader variations will be apparent to those of ordinary skill in the art once aware of the present invention.

The following examples, exemplify, but do not limit, the invention, which is limited only by the claims appended hereto.

### EXAMPLES

### Example 1

A patient on atorvastatin 80 mg once daily is found to still be in need of reducing weight, triglyceride, and cholesterol levels further. 120 mg once daily orlistat is added to his regimen and the patient begins to lower his weight, serum triglycerides, and cholesterol. The patient is maintained on this regimen for 2 months and thereafter the atorvastatin dosage is reduced to 60 mg once daily at which the reductions previously obtained are maintained. The patient is then switched to a fixed combination dosage form of 120 mg orlistat and 60 mg atorvastatin once daily.

### Example 2

A patient on atorvastatin 10 mg once daily is found to be in need of weight reduction and triglyceride reduction, although cholesterol levels are adequately maintained by the atorvastatin. Orlistat 120 mg once daily is added to the regimen and each of weight, triglycerides and cholesterol drop. After 6 weeks on this therapy, the patient is changed to 120 mg orlistat once daily and 5 mg atorvastatin once daily, which surprisingly maintains the lowered weight, triglycerides, and cholesterol levels achieved at the higher atorvastatin dose. The patient is then changed to a fixed combination of 120 mg orlistat and 5 mg of atorvastatin.

### Example 3

### Compositions Containing Atorvastatin Hemicalcium and Orlistat as Active Agents are Prepared as Follows

| Ingredients | Composition 1 | Composition 2 |
|---|---|---|
| Intra-granular | | |
| Atorvastatin Ca * | * | * |
| Croscarmellose Sodium | 48 | 48 |
| Vitamin E TPGS | 20 | 40 |
| MCC PH 102 | — | 162.6 |
| Extra-granular | | |
| Orlistat | 120 | 120 |
| Lactose Monohydrate | 292 | 292 |
| (Pharmatose DCL 11) | | |
| MCC Avicel pH 102 | 269 | 87 |
| Sodium starch glycollate | 48 | 48 |
| Magnesium stearate | 7.2 | 7.2 |
| Coating | | |
| Opadry white | 23 | — |
| Opadry pink | — | 23 |

* EQUIVALENT TO 80 MG OF ATORVASTATIN

Method of Manufacture:

Atorvastatin calcium and croscarmellose sodium (and microcrystalline cellulose in the case of formulation 2) were sifted together and dry blended. Separately, Vitamin E TPGS was dissolved in warm water to obtain a clear solution and used to granulate the dry blend in a high shear mixer. The wet granules were sieved and dried at a product bed temperature of 45-50° C. The dried granules were then sized and mixed with the orlistat and the other inactive ingredients other than the magnesium stearate, and then the magnesium stearate was added. The resulting mixture was then compressed into tablets and the tablets coated with Opadry.

Dissolution:

The dissolution studies were performed on six tablets per each formulation with comparisons made between the two compositions of the invention and LIPITOR (Pfizer) tablets having the same amount of atorvastatin calcium present. The dissolution parameters and release profiles are as set forth below

| | |
|---|---|
| Medium | 0.1N HCl (with 0.2% NaCl) |
| Volume | 900 mL |
| Apparatus | USP Type II (Paddle) |
| Rotation | 50 rpm |
| Quantitification | UV |

| | Batch nos. | | |
|---|---|---|---|
| | Lipitor | | |
| Time | (03967V) | Composition 1 | Composition 2 |
| (Minutes) | Percent drug released | | |
| 5 | 29.2 | 31.8 | 26.9 |
| 10 | 33.3 | 45.1 | 45.9 |
| 15 | 35.8 | 53.8 | 52.1 |
| 30 | 38.6 | 62.2 | 60.3 |
| 45 | 41.3 | 70.8 | 61.7 |
| 60 | 42.6 | 71.8 | 66.7 |

The disslution of atorvastatin in 0.1N HCl was significantly increased compared to the Pfizer product. Since atrovastatin absorbs form the stomach increase in the dissolution in the gastric fluid translates to increased in the bioavailability. Based on the dissolution data one would expect that the bioavailability of our novel formulations will be increased by at least 70%.

### Example 4

Example 3 is repeated except that the Orlistat is blended with the atorvastatin before granulation so that the orlistat is intragranular.

### Example 5-10

Examples 3 and 4 are repeated with the further addition of a non-statin antihypertensive being added as a third active agent. In Examples 5 & 6, the non-statin antihypertensive is added as a further intragranular component along with the atorvastatin, but otherwise the formulation is as in Examples 3 and 4 respectively. In Examples 7 and 8, Examples 3 and 4 are repeated except that the additional non-statin antihypertensive is added extragranularly so that it is not in intimate admixture with the atorvastatin. In Examples 9 and 10, Examples 3 and 4 are repeated except that a separate granulation containing the non-statin antihypertensive is blended with the atorvastatin containing granulate and the extragranulate components before compression.

1. A method of reducing serum triglyceride and/or serum cholesterol in a patient comprising administering as co-therapy both (a) at least one statin and (b) at least one anti-obesity agent.

2. The method of claim 1 wherein the statin is selected from atorvastatin, lovastatin, fluvastatin, pravastatin, rosuvastatin, or simvastatin or a pharmaceutically acceptable salt thereof or a lactone version thereof.

3. The method of claim 1 wherein the statin is atorvastatin or a pharmaceutically acceptable salt thereof.

4. The method of claim 1 wherein the anti-obesity agent is selected from orlistat and a sibutramine-type agent or a pharmaceutically acceptable salt thereof.

5. The method of claim 1 wherein the anti-obesity agent is selected from orlistat or sibutramine or a pharmaceutically acceptable salt thereof.

6. The method of claim 1 further comprising at least one further active agent.

7. The method of claim 1 wherein said at least one further active agent is selected from the group consisting of non-statin antihypertensive agents.

8. The method of claim 1 wherein said co-therapy is achieved by administering a fixed combination dosage form comprising said at least one anti-obesity agent and said at least one statin.

9. The method of claim 8 wherein the anti-obesity agent is selected from orlistat, sibutramine, or a pharmaceutically acceptable salt thereof.

10. The method of claim 8 wherein the statin is atorvastatin or a pharmaceutically acceptable salt thereof.

11. The method of claim 8 wherein said fixed combination dosage form comprises
a statin;
an anti-obesity agent;
croscarmellose sodium;
vitamin E TPGS;
microcrystalline cellulose;
hydrated lactose;
sodium starch glycollate;
magnesium stearate; and
film coating components.

12. The method of claim 11 wherein said fixed combination dosage form further comprises at least one additional active agent selected from non-statin antihypertensive agents.

13. A method of making a fixed combination dosage form of claim 8 comprising
blending at least one of said croscarmellose sodium and said sodium starch glycollate with at least a portion of (a) said statin and/or at least a portion of (b) said anti-obesity agent and optionally a portion of said microcrystalline cellulose to form a first blend; granulating said first blend with an aqueous solution of said vitamin E TPGS to form a first granulate; drying and sieving said first granulate; blending with said first granulate any of said statin and said anti-obesity agent and said microcrystalline cellulose not included into said first granulate, said hydrous lactose, and any of said croscarmellose and said sodium starch glycolate not included in said first granulate, and said magnesium stearate to form a tabletting blend; compressing said tabletting blend to form a tablet; and film coating said tablet.

14. The method of claim 13 wherein said fixed combination includes at least one further active agent selected from the group consisting of non-statin antihypertensive agents.
